(19) United States
(12) Patent Application Publication
(10) Pub. No.: US 2021/0330759 A1
(43) Pub. Date: Oct. 28, 2021

(54) METHODS AND COMPOSITIONS FOR TREATING CYTOKINE STORM, ARDS, AND ACUTE LUNG INJURY USING BETA-GLUCOCEREBROSIDASE

(71) Applicant: The Cleveland Clinic Foundation, Cleveland, OH (US)

(72) Inventors: Xiaoxia Li, Cleveland, OH (US); Junjie Zhao, Cleveland Heights, OH (US); Quanri Zhang, Cleveland, OH (US)

(21) Appl. No.: 17/234,301

(22) Filed: Apr. 19, 2021

Related U.S. Application Data
(60) Provisional application No. 63/013,630, filed on Apr. 22, 2020.

(51) Int. Cl. A61K 38/47

(52) U.S. Cl. CPC A61K 38/47; C12Y 302/01045

(57) ABSTRACT

The present invention relates to methods and compositions for treating a subject with a condition selected from: i) an on-going cytokine storm, ii) acute respiratory distress syndrome (ARDS), and/or iii) acute lung injury (ALI), using a composition comprising β-glucocerebrosidase, or a vector encoding β-glucocerebrosidase. In certain embodiments, the condition is caused by COVID-19 virus infection or other viral infection.

The present application claims priority to U.S. Provisional application Ser. No. 63/013,630, filed Apr. 22, 2020, which is herein incorporated by reference in its entirety.

### FIELD OF THE INVENTION

The present invention relates to methods and compositions for treating a subject with a condition selected from: i) an on-going cytokine storm, ii) acute respiratory distress syndrome (ARDS), and/or iii) acute lung injury (ALI), using a composition comprising β-glucocerebrosidase, or a vector encoding β-glucocerebrosidase. In certain embodiments, the condition is caused by COVID-19 virus infection or other viral infection.

### BACKGROUND OF THE INVENTION

β-glucosylceramide is a glycosphingolipid widely found in human tissues. It is composed of a saccharide chain and a ceramide in which a fatty acid chain is attached to the platform sphingosine. As an integral part of cell membrane, it is crucial for a variety of cellular processes including lipid raft formation and importantly, endocytosis. β-GluCer synthesis is catalyzed by UDP-glucose ceramide glucosyltransferase through glycosylation of ceramide and its degradation occurs in lysosome, catalyzed by the rate-limiting enzyme called glucocerebrosidase (GBA). Of interest, glucosylceramide synthase was shown to be required for the entry and infection of influenza virus, revealing a role of β-GluCer in mediating viral replication.

The turnover of β-GluCer is tightly controlled in physiological condition. Accumulation of β-GluCer is associated with pathologies characterized by chronic inflammation. The most well-known condition that is caused by β-GluCer accumulation is Gaucher's disease, a genetic defect that lead to impaired GBA. GBA1 mutations drive extensive accumulation of glucosylceramide (GC) in multiple innate and adaptive immune cells in the spleen, liver, lung and bone marrow, often leading to chronic inflammation.

### SUMMARY OF THE INVENTION

The present invention relates to methods and compositions for treating a subject with a condition selected from: i) an on-going cytokine storm, ii) acute respiratory distress syndrome (ARDS), and/or iii) acute lung injury (ALI), using a composition comprising β-glucocerebrosidase, or a vector encoding β-glucocerebrosidase. In certain embodiments, the condition is caused by COVID-19 virus infection or other viral infection.

In some embodiments, provided herein are methods of treating a subject comprising: administering or providing a composition to a subject, wherein the composition comprises β-glucocerebrosidase, or a vector encoding β-glucocerebrosidase, and wherein the subject a condition selected from: i) an on-going cytokine storm, ii) acute respiratory distress syndrome (ARDS), and/or iii) acute lung injury (ALI).

In certain embodiments, provided herein are in vitro compositions comprising: a) β-glucocerebrosidase, or a vector encoding β-glucocerebrosidase, and b) a respiratory virus. In further embodiments, the in vitro compositions further comprise human cells. In other embodiments, the respiratory virus comprises a Coronavirus. In other embodiments, the Coronavirus comprises Sars-CoV-2 or Sars-CoV-2.

In certain embodiments, the condition is caused by COVID-19 virus infection. In other embodiments, the subject is a human. In further embodiments, the condition is caused by SARSr-CoV. In other embodiments, the condition is caused by a virus. In further embodiments, the virus is a coronavirus. In some embodiments, the virus is influenza virus. In additional embodiments, the virus is a virus that causes respiratory illness. In particular embodiments, the composition is in the form of an aqueous solution and wherein the administering is via intravenous injection into the subject. In certain embodiments, the administering is via the subject's airway. In other embodiments, the composition is administered via the subject's airway using a nebulizer or inhaler.

In some embodiments, provided herein are systems and kits comprising: a) an airway administration device, and b) a composition comprising β-glucocerebrosidase, or a vector encoding β-glucocerebrosidase. In further embodiments, the airway administration device comprises an inhaler or nebulizer. In certain embodiments, the composition is present inside said airway administration device.

### DESCRIPTION OF THE DRAWINGS

FIG. 1. Treating COVID19 with glucocerebrosidase. Impaired respiratory capacity from virus infection leads to hypoxia, which upregulates the synthesis of β-glucosylceramide. Virus replication also induces cell death, resulting in subsequent release of β-glucosylceramide. β-glucosylceramide is an endogenous ligand recognized by a variety of immune cells, including macrophages, dendritic cells and neutrophils, serving as a danger signal to indicate tissue damage. Upon recognition of β-glucosylceramide, immune cells are activated to produce copious amount of cytokine, engendering cytokine storm and further tissue damage. This cycle results in a feedforward loop that amplifies lung injury and sustains cytokine production, culminating in cytokine storm and multi-organ failure in ARDS. Glucocerebrosidase is administered to a COVID-19 infected subject to reduce the cytokine storm and/or ARDS and acute lung injury.

### DEFINITIONS

As used herein, the terms “host,” “subject” and “patient” refer to any animal, including but not limited to, human and non-human animals (e.g., dogs, cats, cows, horses, sheep, poultry, fish, etc.) that is studied, analyzed, tested, diagnosed or treated. As used herein, the terms “host,” “subject” and “patient” are used interchangeably, unless indicated otherwise. In certain embodiments, the subject is a human (e.g., a human with a viral infection, such as COVID-19 infection or a human that otherwise has ARDS or ongoing cytokine storm or acute lung injury).

### DETAILED DESCRIPTION OF THE INVENTION

The present invention relates to methods and compositions for treating a subject with a condition selected from: i) an on-going cytokine storm, ii) acute respiratory distress syndrome (ARDS), and/or iii) acute lung injury (ALI), using a composition comprising β-glucocerebrosidase, or a vector encoding β-glucocerebrosidase. In certain embodiments, the condition is caused by COVID-19 virus infection or other viral infection.

Provided here is treatments using glucocerebrosidase, or a vector encoding glucocerebrosidase, for treating a subject with a cytokine storm, acute respiratory distress syndrome (ARDS) and/or acute lung injury (ALI), which are critical conditions that frequently culminate in death. Diseases in which aforementioned critical conditions may arise include, but are not limited to, viral infection-induced disease, such as SARS, MERS, COVID19, and flu; bacterial or fungal pneumonia; and therapy-induced cytokine storm and multi-organ failure such those seen in CAR-T therapies; lung injuries resulted from inhalation of smoke or toxic gas; and transfusion-related acute lung injury (TRALI).

There is no proven therapeutics that has demonstrated efficacy in preventing mortality in patients undergoing cytokine storm and ARDS, especially for patients with COVID19. In clinical practice, anti-IL-6 biologics are currently being used off label to treat patients whose serum cytokines register an exorbitant levels. At the time the cytokine storm sets in, multi-organ failure can reach an irreversible point, rendering therapeutic intervention aiming at controlling the cytokine storm futile.

The use of glucocerebrosidase, or a vector encoding glucocerebrosidase may be superior to current therapy as it targets an upstream biological event leading to the onset of cytokine storm and subsequent ARDS and multi-organ failure. Therefore, the intervention described by the current invention can prevent cytokine storm and multi-organ failure.

Gaucher's disease patients were shown to exhibit pulmonary manifestations, described as ground-glass opacity in CT scan. This radiologic feature is similar to the findings from patients with severe viral infection-associated pneumonia (e.g. COVID19). Gaucher's disease can be effectively treated by enzyme replacement therapy using recombinant human GBA, which removes the deposited β-GluCer. In addition to Gaucher's disease, accumulation of β-GluCer is also found in ischemic tissues. The synthesis of β-GluCer was shown to be regulated by O₂ saturation, with hypoxic condition promoting the production of β-GluCer. Relevant to this regulatory mode, β-GluCer level is increased in the lung in response to stress. Bronchoalveolar lavage fluid of elastase-exposed mice (lung injury) showed elevated level of β-GluCer. Consistently, elevated level of β-GluCer is found in the bronchoalveolar lavage fluid of pneumonia patients undergoing acute respiratory distress syndrome. Additionally, obesity has been shown to increase the level of β-GluCer.

Mechanistically, β-GluCer is released by dying cells. Extracellular β-GluCer can be recognized by immune cells expressing a C-type lectin receptor, MINCLE. Activation of myeloid MINCLE pathway initiates NF-kB signaling and promotes the cytokine production, including IL-6, TNFa and IL-1β. In work conducted during development of the present invention, it was shown that β-GluCer can activate Mincle on T cells to drive Th17 polarization and drive Th17-mediated inflammation. As a result, increased level of β-GluCer can amplify inflammatory responses, leading to massive cell death and tissue damage.

Cytokine storm, or cytokine release syndrome is a fatal pathogenic event commonly seen in patients with sepsis, viral infection or drug reaction. The most famous disease where cytokine storm has been noted is COVID19, which is caused by the pathogen SARS-CoV2, a novel coronavirus. Representing an immune overdrive, cytokine storm often culminates in multi-organ failure and death. The trigger of cytokine storm varies from condition to condition. However, a feedforward loop that sustains and amplifies the production/release of cytokines is required to enable the production of copious amount of cytokine. Breaking the feedforward loop is required for attenuating the cytokine storm. Acute respiratory distress syndrome (ARDS) is a type of respiratory failure characterized by rapid onset of widespread inflammation in the lungs. ARDS is the leading cause of mortality in viral infection-induced diseases, including COVID19, SARS, MERS and flu. Known risk factor for ARDS include old age and overweight.

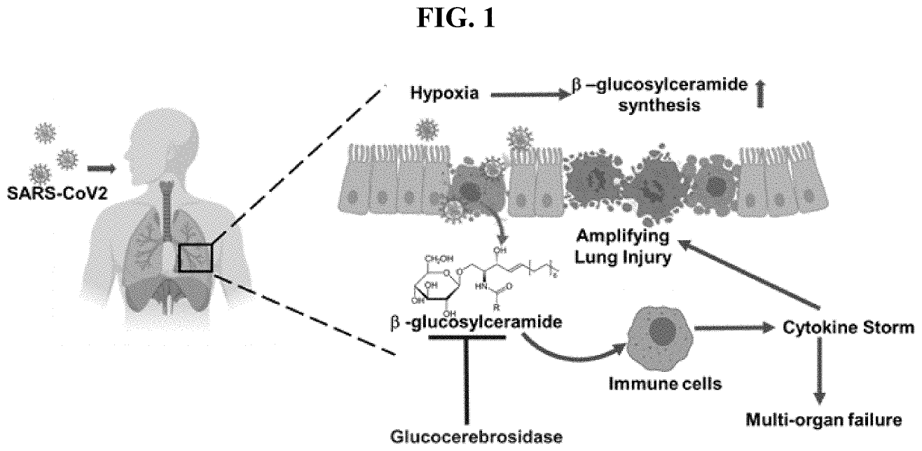

In certain embodiments, glucocerebrosidase, or a vector encoding glucocerebrosidase, are used administered to subject with COVID-19 infection or other respiratory virus infection. As shown in the hypothetical model in FIG. 1, impaired respiratory capacity from virus infection leads to hypoxia, which upregulates the synthesis of β-glucosylceramide. Virus replication also induces cell death, resulting in subsequent release of β-glucosylceramide. β-glucosylceramide is an endogenous ligand recognized by a variety of immune cells, including macrophages, dendritic cells and neutrophils, serving as a danger signal to indicate tissue damage. Upon recognition of β-glucosylceramide, immune cells are activated to produce copious amount of cytokine, engendering cytokine storm and further tissue damage. This cycle results in a feedforward loop that amplifies lung injury and sustains cytokine production, culminating in cytokine storm and multi-organ failure in ARDS.

In certain embodiments, provided here methods to treat cytokine storm and ARDS using recombinant glucocerebrosidase or a vector encoding glucocerebrosidase. In particular embodiments, the method involve infusion of a recombinant human glucocerebrosidase into patients at risk for or suffering from cytokine storm or ARDS. In certain embodiments, patients with the following conditions may be treated with the current invention: Viral infection-induced disease (e.g., SARS, MERS, COVID19, and flu), bacterial or fungal pneumonia; therapy-induced cytokine storm and multi-organ failure such those seen in CAR-T therapies; lung injuries resulted from inhalation of smoke or toxic gas; and transfusion-related acute lung injury (TRALI).

In one example, glucocerebrosidase, or a vector encoding glucocerebrosidase, is used to treat COVID-19 infection as shown in FIG. 1. COVID19 patients with mild symptoms who are deemed to be at a high risk of progression may be treated with infusion of recombinant glucocerebrosidase to prevent disease progression. The method of determining the risk for disease aggravation can be based on the attending physician's clinical assessment or based on a laboratory assay that determines (1) the level of β-GluCer in the blood or bronchoalveolar lavage fluid; and/or (2) serum cytokine levels. COVID19 patients with severe symptoms can be treated with infusion of recombinant glucocerebrosidase to prevent ARDS or cytokine release syndrome. COVID19 patients in critical condition may be treated with infusion of recombinant glucocerebrosidase as a rescuing therapy.

### REFERENCES

1. van Meer, G., J. Wolthoorn, and S. Degroote, The fate and function of glycosphingolipid glucosylceramide. Philos Trans R Soc Lond B Biol Sci, 2003. 358(1433): p. 869-73.
2. Messner, M. C. and M. C. Cabot, Glucosylceramide in humans. Adv Exp Med Biol, 2010. 688: p. 156-64.
3. Ichikawa, S. and Y. Hirabayashi, Glucosylceramide synthase and glycosphingolipid synthesis. Trends Cell Biol, 1998. 8(5): p. 198-202.
4. Rijnboutt, S., et al., Mannose 6-phosphate-independent membrane association of cathepsin D, glucocerebrosidase, and sphingolipid-activating protein in HepG2 cells. J Biol Chem, 1991. 266(8): p. 4862-8.
5. Nagata, M., et al., Intracellular metabolite beta-glucosylceramide is an endogenous Mincle ligand possessing immunostimulatory activity. Proc Natl Acad Sci USA, 2017. 114(16): p. E3285-e3294.
6. Aerts, J. M., et al., Pharmacological inhibition of glucosylceramide synthase enhances insulin sensitivity. Diabetes, 2007. 56(5): p. 1341-9.
7. Kurz, J., et al., The relevance of ceramides and their synthesizing enzymes for multiple sclerosis. Clin Sci (Lond), 2018. 132(17): p. 1963-1976.
8. Nilsson, O. and L. Svennerholm, Accumulation of glucosylceramide and glucosylsphingosine (psychosine) in cerebrum and cerebellum in infantile and juvenile Gaucher disease. J Neurochem, 1982. 39(3): p. 709-18.
9. Hruska, K. S., et al., Gaucher disease: mutation and polymorphism spectrum in the glucocerebrosidase gene (GBA). Hum Mutat, 2008. 29(5): p. 567-83.
10. Santamaria, F., et al., Pulmonary manifestations of Gaucher disease: an increased risk for L444P homozygotes? Am J Respir Crit Care Med, 1998. 157(3 Pt 1): p. 985-9.
11. Goitein, O., et al., Lung involvement and enzyme replacement therapy in Gaucher's disease. Qjm, 2001. 94(8): p. 407-15.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A method of treating a subject comprising:
administering or providing a composition to a subject,
wherein said composition comprises β-glucocerebrosidase, or a vector encoding β-glucocerebrosidase, and
wherein said subject a condition selected from: i) an on-going cytokine storm, ii) acute respiratory distress syndrome (ARDS), and/or iii) acute lung injury (ALI).

2. The method of claim 1, wherein said condition is caused by COVID-19 (SARS-CoV-2) virus infection.

3. The method of claim 1, wherein said subject is a human.

4. The method of claim 1, wherein said condition is caused by SARS-CoV-1.

5. The method of claim 1, wherein said condition is caused by a virus.

6. The method of claim 5, wherein said virus is a coronavirus.

7. The method of claim 5, wherein said virus is influenza virus.

8. The method of claim 5, wherein said virus is a virus that causes respiratory illness.

9. The method of claim 1, wherein said composition is in the form of an aqueous solution and wherein said administering is via intravenous injection into said subject.

10. The method of claim 1, wherein said administering is via said subject's airway.

11. The method of claim 1, wherein said composition is administered via said subject's airway using a nebulizer or inhaler.

12. An in vitro composition comprising:
a) β-glucocerebrosidase, or a vector encoding β-glucocerebrosidase, and
b) a respiratory virus.

13. The in vitro composition of claim 12, further comprising human cells.

14. The in vitro composition of claim 12, wherein said respiratory virus comprises a Coronavirus.

15. The in vitro composition of claim 14, wherein said Coronavirus comprises Sars-CoV-2 or Sars-CoV-2.

16. A system or kit comprising:
a) an airway administration device, and
b) a composition comprising β-glucocerebrosidase, or a vector encoding β-glucocerebrosidase.

17. The system or kit of claim 16, wherein said airway administration device comprises an inhaler or nebulizer.

18. The system or kit of claim 16, wherein said composition is present inside said airway administration device.
